(19) United States
(12) Patent Application Publication
(10) Pub. No.: US 2024/0139154 A1
(43) Pub. Date: May 2, 2024

(54) Methods of Reducing Risk of Prostate Cancer Progression

(71) Applicant: Astellas Pharma Inc., Tokyo (JP)

(72) Inventors: John Hairston, Northbrook, IL (US); Bruce Brown, Northbrook, IL (US)

(21) Appl. No.: 18/279,103

(22) PCT Filed: Feb. 28, 2022
(86) PCT No.: PCT/JP22/08383

§ 371 Date: Aug. 28, 2023

Related U.S. Application Data
(60) Provisional application No. 63/222,323, filed on Jul. 15, 2021.
(60) Provisional application No. 63/154,426, filed on Feb. 26, 2021.

(51) Int. Cl. A61K 31/4166; A61K 45/06; A61P 35/00

(52) U.S. Cl. CPC A61K 31/4166; A61K 45/06; A61P 35/00

(57) ABSTRACT

This disclosure provides methods of reducing the risk of prostate cancer progression in patients with a histologically proven prostate cancer characterized as low or intermediate risk for which no other treatment for prostate cancer is indicated. The method comprises administering enzalutamide as a monotherapy in an amount and for a period of time clinically proven effective in reducing the risk of prostate cancer progression in a control population of patients under active surveillance and having histologically proven prostate cancers characterized as low or intermediate risk for which no other treatment for prostate cancer is indicated.

### CROSS REFERENCE

Each reference cited herein is incorporated by reference in its entirety.

### TECHNICAL FIELD

This disclosure relates generally to treatment of prostate cancer.

### BRIEF DESCRIPTION OF DRAWINGS

FIG. 1 is a chart showing the disposition of patients.

FIG. 2 is a Kaplan-Meier Plot of Time to Disease Progression (full analysis set). Footnotes to FIG. 2: ^{a}Pathological progression was defined as an increase in primary or secondary GS by >1 or >15% increase in cancer-positive cores; ^{b} Therapeutic progression was defined as the earliest occurrence of primary therapy for prostate cancer (prostatectomy, radiation, focal therapy, or systemic therapy); ^{c}Patients with no prostate cancer progression at the time of study completion, patients who discontinued, and patients who died were censored at the last assessment date. Patients switching therapy during the study were censored at the time of the initial therapy switch, and patients discontinuing therapy were censored at the time of study discontinuation; ^{d}Calculated using a two-sided, log-rank test; ^{e}Calculated using a Cox regression model assuming proportional hazards with treatment group, stratification factors, age, race, and time since prostate cancer diagnosis as fixed effects, and study site and patient as random effects. HR<1 favors enzalutamide; ^{f}Calculated using a two-sided, stratified, log-rank testAS, active surveillance; CI, confidence interval; GS, Gleason Score; HR, hazard ratio; NR, not reached.

FIG. 3 is a Kaplan-Meier Port of Time to Prostate Specific Antigen (PSA) Progression. Footnotes to FIG. 3: ^{a}PSA progression was defined as a secondary rise in serum PSA ≥25% of baseline, or ≥25% above nadir, or an absolute increase of ≥2 ng/mL. Patients with no PSA progression at the time of study completion, patients who discontinued, and patients who died were censored at the last assessment date. Patients switching therapy during the study were censored at the time of the initial therapy switch, and patients discontinuing therapy were censored at the time of study discontinuation; ^{b}Calculated using a two-sided, log-rank test; ^{c}Calculated using a Cox regression model assuming proportional hazards with treatment group, stratification factors, age, race, and time since prostate cancer diagnosis as fixed effects, and study site and patient as random effects. HR<1 favors enzalutamide; ^{d}Calculated using a two-sided, stratified, log-rank test. AS, active surveillance; CL, confidence interval; HR, hazard ratio; PSA, prostate-specific antigen.

### BACKGROUND

For some prostate cancer patients, active surveillance or watchful waiting can delay treatment of prostate cancer or prevent unnecessary treatment. However, some patients eventually do experience progression of their prostate cancers. It would therefore be advantageous to have a method of reducing the risk of prostate cancer progression in patients under active surveillance or watchful waiting.

### DETAILED DESCRIPTION

This disclosure provides a method of reducing the risk of prostate cancer progression. The method comprises administering enzalutamide as a monotherapy for low or intermediate risk prostate cancer. Enzalutamide (marketed as XTANDI®) is a second generation nonsteroidal androgen receptor inhibitor currently approved for treating castration-resistant prostate cancer and metastatic castration-sensitive prostate cancer. The approved dosage is 160 mg (two 80 mg tablets or four 40 mg tablets or four 40 mg capsules) administered orally once daily. Enzalutamide is disclosed in U.S. Pat. Nos. 8,183,274; 7,709,517; and 9,126,941, each of which is incorporated by reference in its entirety.

“Monotherapy” (or “monotherapy for the prostate cancer”) as used in this disclosure means the only therapy administered to treat the prostate cancer and does not exclude therapy for other purposes.

Patients suitable for this enzalutamide monotherapy have a histologically proven prostate cancer characterized as low or intermediate risk for which no other treatment for prostate cancer is indicated, i.e., patients under active surveillance (AS) or watchful waiting. “Active surveillance” and “watchful waiting” as used in this disclosure mean periodically monitoring a patient's condition (e.g., by prostate-specific antigen (PSA) blood tests, digital rectal exams, biopsies, and/or imaging tests) but not administering any treatment for the prostate cancer.

Unless defined elsewhere in this disclosure, “low risk” is defined as one or more of stage T1c-T2a, PSA level <10 ng/mL, no positive lymph nodes (NO), no metastases (MO) (or presumed NO and MO if a CT/bone scan was not done), Gleason score (GS) 6, and Eastern Cooperative Oncology Group (ECOG) status of <2.

Unless defined elsewhere in this disclosure, “intermediate risk” is defined as one or more of stage T2b-T2c, PSA level <20 ng/mL, NO, MO (or presumed NO and MO if a CT/bone scan was not done), GS<7 (3+4 pattern), and an ECOG status <2.

In some embodiments, the life expectancies of the patient and patients in the control population are >5 years; i.e., patients who otherwise would be under active surveillance. In some embodiments, the life expectancies of the patient and patients in the control population are <5 years; i.e., patients who otherwise may be under active surveillance or under watchful waiting.

The enzalutamide monotherapy typically is administered orally. Enzalutamide is typically administered orally at a daily dose of 160 mg. For patients to whom a strong CYP3A4 inducer is co-administered, the enzalutamide monotherapy is administered orally at a daily dose of 240 mg. In some embodiments, enzalutamide monotherapy is administered locally at the site of the tumor, e.g., by intratumoral injection or by using, for example, an implanted device or sustained release system.

The enzalutamide monotherapy is administered for a period of time sufficient to reduce the risk of prostate cancer progression (e.g., 30, 60, 90 days; 3, 6, 9, 12, 18, 24 months).

As shown in the Example below, enzalutamide monotherapy is well-tolerated and has several clinically proven advantages.

First, enzalutamide monotherapy is effective in reducing the risk of prostate cancer progression by 46% compared to a control population of patients under surveillance having histologically proven prostate cancers characterized as low or intermediate risk for which no other treatment for prostate cancer is indicated, but not receiving any treatment for prostate cancer. “Prostate cancer progression” can be pathological prostate cancer progression, therapeutic prostate cancer progression, or both. “Pathological prostate cancer progression” is characterized by an increase in primary or secondary Gleason pattern by >1 or a >15% increase in cancer positive cores. “Therapeutic prostate cancer progression” is the earliest occurrence of a further therapy for prostate cancer, such as androgen deprivation therapy, prostatectomy, radiation, focal therapy, and systemic therapy.

Second, compared to the control population, patients receiving enzalutamide monotherapy were at least three times more likely to have a negative biopsy at one year.

Third, patients receiving enzalutamide monotherapy had PSA progression delayed by six months. “PSA progression” is defined as a ≥25% increase in serum PSA from baseline, a ≥25% increase above nadir, or an absolute increase of ≥2 ng/mL.

Other prostate cancer monotherapies can be used in the disclosed methods; that is, this disclosure also provides a method of reducing the risk of prostate cancer progression in a patient with a histologically proven prostate cancer characterized as low or intermediate risk for which no other treatment for prostate cancer is indicated, comprising administering a prostate cancer monotherapy to the patient in an amount and for a period of time clinically proven effective in reducing the risk of prostate cancer progression relative to a control population of patients under surveillance, wherein patients in the control population have histologically proven prostate cancers characterized as low or intermediate risk for which no other treatment for prostate cancer is indicated, and wherein patients in the control population does not receive a therapy for prostate cancer.

In some embodiments, the prostate cancer monotherapy is a second-generation nonsteroidal androgen receptor such as abiraterone acetate, apalutamide, or darolutamide. Abiraterone acetate typically is administered orally at a dose of 1,000 mg once a day together with 5 mg prednisone twice a day; accordingly, “abiraterone acetate monotherapy” includes administration of both abiraterone acetate and prednisone. Apalutamide typically is administered orally at a dose of 240 mg daily. Darolutamide typically is administered orally at a daily dose of 600 mg.

This disclosure also provides a method of treating low or intermediate risk prostate cancer by administering to a patient having a low or intermediate risk prostate cancer a therapeutically effective amount of enzalutamide, abiraterone acetate, apalutamide, or darolutamide as a monotherapy. In one embodiment, the method comprises administering enzalutamide as a monotherapy. In one embodiment, the method comprises administering abiraterone acetate as a monotherapy, wherein “abiraterone acetate monotherapy” includes administration of both abiraterone acetate and prednisone. In one embodiment, the method comprises administering apalutamide as a monotherapy. In one embodiment, the method comprises administering darolutamide as a monotherapy.

### Example 1

Study Overview

A description of the ENACT study, NCT02799745, was posted on the website clinicaltrials.gov on Jun. 15, 2016, and that description is incorporated herein by reference. The primary objective of the study was to evaluate the efficacy and safety of enzalutamide for extension of time to prostate cancer progression (pathological or therapeutic) in patients with histologically proven prostate cancer that was categorized as low risk or intermediate risk, who were under AS, and who had not received prior local or systemic prostate cancer therapy.

Study Population

Eligible patients were diagnosed within 6 months of screening and were under AS. Patients with clinically localized, histologically proven prostate cancer that was categorized as low risk or intermediate risk and who were under AS were randomized 1:1 to receive treatment with 160 mg/day enzalutamide (N=114) or active surveillance (N=113) for one year or until pathological or therapeutic progression. The average age was 66.1 years at baseline.

Study Design and Methodology

Patients were stratified by low versus intermediate risk and type of biopsy performed (multi-parametric magnetic resonance imaging (mpMRI) targeted versus non mpMRI targeted transrectal ultrasound-guided prostate biopsy). “Low risk” was defined as T1c-T2a, PSA <10 ng/mL, NO, MO, GS ≤6, ECOG status ≤2, and estimated life expectancy >5 years. “Intermediate risk” was defined as T2b-T2c, PSA <20 ng/mL, NO, MO, GS ≤7 (3+4 pattern only), ECOG status ≤2, and estimated life expectancy >5 years.

Following randomization to either the enzalutamide group or the AS group, patients were followed for one year (Treatment Period). This was followed by a one year follow up period, and a further continued follow-up period of at least one-year, until the last patient completed the 24-month visit. Biopsies were evaluated through a blinded central review.

Primary and Selected Secondary Endpoints

The primary endpoint was time to prostate cancer progression (pathological or therapeutic progression). “Prostate cancer progression” is defined as pathological or therapeutic prostate cancer progression. “Pathological progression” is defined as an increase in primary or secondary Gleason pattern by >1 or >15% increase in cancer positive cores. “Therapeutic progression” is defined as the earliest occurrence of primary therapy for prostate cancer (e.g., prostatectomy, radiation, focal therapy, or systemic therapy).

Secondary endpoints were (1) Incidence of negative biopsies for cancer at 1 year and 2 years; (2) percentage of cancer-positive cores at 1 year and 2 years; (3) time to PSA progression; and (4) incidence of a secondary rise in serum PSA at 1 year and 2 years. “PSA progression” is defined as a ≥25% increase in serum PSA from baseline, a ≥25% increase above nadir, or an absolute increase of ≥2 ng/mL.

Statistical Methodology

The sample size calculation assumed a study duration of three years, a loss to follow-up of 16%, an assumed hazard ratio of 0.52, and a three-year median time-to-progression for the control group (Hazard rate=0.2310); this resulted in 72 events achieving 80% power.

The full analysis set (FAS) was the patients who were enrolled in the study and randomized to one or the other of the study arms. The safety set (SAF) was redefined in the Statistical Analysis Plan (SAP) to include only subjects who took the study drug. The FAS was 227 patients (113 in the active surveillance arm, 114 in the enzalutamide arm). The SAF had 112 subjects in the enzalutamide arm.

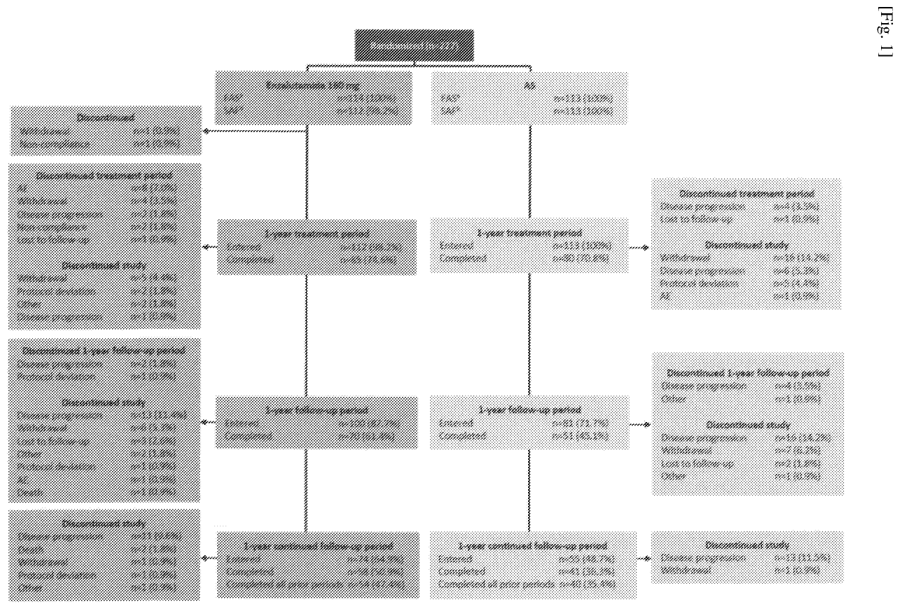

In total, 94 men completed all study periods (enzalutamide, n=54; AS, n=40; FIG. 1). Baseline demographics and disease characteristics were similar between treatment arms (Table 1).

In total, 121 (53.3%) men had low-risk prostate cancer and 172 (75.8%) men underwent non-mpMRI-targeted biopsy (Table 1). Median enzalutamide treatment duration was 352 days (range 1-393).

TABLE 1
Baseline demographics and disease characteristics

| | Enzalutamide | | | |
|---|---|---|---|---|
| | 160 mg | | AS | |
| Parameter | (n = 114) | | (n = 113) | |
| Mean age ± SD, years | 65.2 ± 8.2 | | 66.9 ± 7.3 | |
| Mean BMI ± SD, km/m² | 29.7 ± 4.4 | | 28.7 ± 4.4 | |
| Prostate cancer risk, n (%) | | | | |
| Low | 61 | (53.5) | 60 | (53.1) |
| Intermediate | 53 | (46.5) | 53 | (46.9) |
| Type of biopsy, n (%) | | | | |
| mpMRI targeted | 27 | (23.7) | 28 | (24.8) |
| Non-mpMRI targeted | 87 | (76.3) | 85 | (75.2) |
| Clinical tumor stage at prostate | | | | |
| cancer diagnosis, n (%) | | | | |
| T1 | 0 | | 1 | (0.9) |
| T1a | 0 | | 0 | |
| T1b | 0 | | 0 | |
| T1c | 90 | (78.9) | 89 | (78.8) |
| T2 | 1 | (0.9) | 2 | (1.8) |
| T2a | 16 | (14.0) | 15 | (13.3) |
| T2b | 3 | (2.6) | 3 | (2.7) |
| T2c | 4 | (3.5) | 3 | (2.7) |
| Clinical lymph nodes at prostate | | | | |
| cancer diagnosis, n (%) | | | | |
| NX | 50 | (43.9) | 42 | (37.2) |
| N0 | 64 | (56.1) | 71 | (62.8) |
| Distant metastases at prostate | | | | |
| cancer diagnosis, n (%) | | | | |
| MX | 53 | (46.5) | 48 | (42.5) |
| M0 | 61 | (53.5) | 65 | (57.5) |
| Median (range) time since prostate | 0.2 | (0-5) | 0.2 | (0-2) |
| cancer diagnosis, years | | | | |
| Total Gleason score at prostate | | | | |
| cancer diagnosis, n (%) | | | | |
| 6 | 67 | (58.8) | 66 | (58.4) |
| 7 (3 + 4 pattern only) | 46 | (40.4) | 47 | (41.6) |
| Unknown | 1 | (0.9) | 0 | |
| LS mean cancer positive cores ± SE^{a}, % | 25.43 ± 1.61 | | 23.12 ± 1.58 | |
| Median (range) serum PSA, ng/mL | 5.8 | (1-17) | 5.9 | (1-23) |

^{a}Based on the most recent biopsy taken during the 6 months prior to screening
AS, active surveillance; BMI, body mass index; LS, least squares; mpMRI, multiparametric magnetic resonance imaging; PSA, prostate-specific antigen; SD, standard deviation; SE, standard error.

All statistical comparisons were made using 2-sided tests at α=0.05 significance level and 95% confidence intervals.

The primary efficacy endpoint of time to prostate cancer progression (pathological or therapeutic) was analyzed using Kaplan-Meier (KM) methods, where median and 95% confidence interval for time to disease progression were calculated for each treatment group. Patients were censored if they had no disease progression. Discontinuation or death were censored at discontinuation date or last assessment date. Additionally, patients who switched therapy during the study were censored at the time of the initial therapy switch. Hazard ratio (enzalutamide/AS) and 95% confidence interval were calculated based on Cox regression models with treatment groups, stratification factors, age, race, and time since prostate cancer diagnosis as fixed factors; and site as random effects to test the hypothesis that time to disease progression between the two groups would not be the same.

Secondary endpoint of incidence of negative biopsy for cancer was summarized using frequencies and percentages at one and two years by treatment groups. Comparison between the enzalutamide and AS groups was calculated from an exact logistic regression using the treatment groups, randomization stratification factors [prostate cancer risk (low/intermediate), type of biopsy performed (mpMRI targeted/non mpMRI targeted)], age, race, and time since prostate cancer diagnosis as fixed effects and site and subject as random effects. Analysis of time to PSA progression was performed using the same method used to analyze the primary endpoint. P-values were adjusted using the Bonferroni-Holm method to control type I error at 0.05 for selected secondary endpoints.

Study Results

Subject Disposition

The 227 enrolled patients were randomized into the enzalutamide arm (114 patients) or the AS arm (113 patients). Overall, 165 patients completed the one-year treatment period, 70.8% (80/113) in the AS arm and 74.6% (85/114) in the enzalutamide arm. One hundred seventeen patients completed the one-year follow-up period, 45.1% (51/113) in the AS arm and 57.9% (66/114) in the enzalutamide arm. A total of 94 patients completed all study periods, 35.4% (40/113) in the AS arm and 47.4% (54/114) in the enzalutamide arm.

Demographic and Baseline Characteristics

Ethnicity, race and age categories were similar across the two treatment groups. Baseline disease characteristics were similar across the treatment groups.

Treatment Exposure

Throughout the one-year treatment period, the mean (SD) treatment duration was 300.1 (112.73) days for 112 of treated enzalutamide patients in the FAS. The percentage of patients who had a dose decrease was 12.3% (14/114), with the primary reason being adverse events, 2.6% (3/114) had a dose increase, and 13.2% (15/114) had a dose interruption. Patients did not receive the study drug during the one-year follow-up or the one-year continued follow-up periods.

### Primary Efficacy Endpoint

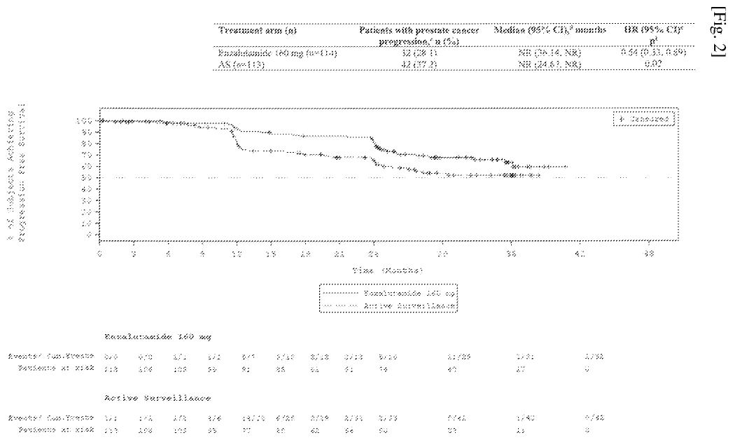

The Kaplan-Meier curve for time to disease progression is shown in FIG. 2. The results favor the enzalutamide group over the active surveillance group. Median time to pathological or therapeutic prostate cancer progression was not reached in either treatment arm. Enzalutamide reduced the risk of prostate cancer by 46%, which is significant.

Of the 227 patients, 28.1% (32/114) from the enzalutamide group developed a disease progression, while 37.2% (42/113) from the active surveillance group progressed. The median progression free survival for each of the treatment groups was not reached as shown in Table 2. The hazard ratio (95% CI) was 0.542 (0.330, 0.892), favoring enzalutamide vs. active surveillance patients with a statistically significant P-value of 0.016.

TABLE 2
Kaplan-Meier and Cox Regression for
Progression Free Survival -FAS

| | enzalutamide | Active Surveillance |
|---|---|---|
| Analysis Set = FAS | N = [114] | N = [113] |
| No. of Events -n (%) | 32 (28.1) | 42 (37.2) |
| No. Censored - n (%) [1] | 82 (71.9) | 71 (62.8) |
| Median Progression Free | NR (36.14, | NR (24.67, |
| Survival (95% CI) | NR) | NR) |

| | |
|---|---|
| Hazard Ratio (95% CI) [2] | 0.542 (0.330, 0.892) |
| P-Value [3] | 0.016 |

[1] Patients with no cancer progression at the time of study completion, discontinuation or death were censored at the last assessment date. Additionally, patients switching therapy during the study were censored at the time of the initial therapy switch, and patients discontinuing therapy were censored at the time of study discontinuation.
[2] Hazard ratio and 95% CI for hazard ratio are based on a Cox regression model with treatment, prostate cancer risk (low vs. intermediate) and type of biopsy (mpMRI targeted vs. non mpMRI targeted), baseline variables including age, race and time since prostate cancer diagnosis as fixed effects and random effect of site.
[3] P-value is from a 2-sided, log-rank test.

Secondary Efficacy Endpoints

Table 3 summarizes the secondary efficacy endpoints.

TABLE 3
Secondary efficacy endpoints

| | Enzalutamide | | | |
|---|---|---|---|---|
| Endpoint | 160 mg | | AS | |
| Incidence of negative biopsy at 1 year | n = 114 | | n = 113 | |
| Biopsy result, n (%) | | | | |
| Negative | 40 | (35.1) | 16 | (14.2) |
| Positive | 53 | (46.5) | 74 | (65.5) |
| Unknown | 21 | (18.4) | 23 | (20.4) |
| OR^{a} (95% CI)^{b} | 3.5 (1.76, 6.92) | | | |
| p^{c} | <0.0001 | | | |
| Incidence of negative biopsy at 2 years | n = 100 | | n = 83 | |
| Biopsy result, n (%) | | | | |
| Negative | 19 | (19.0) | 10 | (12.0) |
| Positive | 47 | (47.0) | 40 | (48.2) |
| Unknown | 34 | (34.0) | 33 | (39.8) |
| OR (95% CI)^{a} | 1.6 (0.66, 4.00) | | | |
| p^{c} | 0.29 | | | |
| Percentage of cancer-positive cores | n = 90 | | n = 81 | |
| at 1 year^{d} | | | | |
| LS mean change from baseline ± SE^{e} | −12.97 ± 1.99 | | −2.90 ± 2.03 | |
| (95% CI) | (−16.91, −9.03) | | (−6.93, 1.12) | |
| Difference in LS means ± SE | −10.07 ± 2.40 | | | |
| (95% CI) | (−14.79, −5.34) | | | |
| p^{f} | <0.0001 | | | |
| Percentage of cancer-positive cores | n = 53 | | n = 42 | |
| at 2 years^{d} | | | | |
| LS mean change from baseline ± SE^{e} | −6.68 ± 2.37 | | −1.53 ± 2.57 | |
| (95% CI) | (−11.36, −2.00) | | (−6.61, 3.55) | |
| Difference in LS means ± SE | −5.15 ± 3.17 | | | |
| (95% CI) | (−11.40, 1.11) | | | |
| p^{f} | 0.11 | | | |
| Incidence of a secondary rise in serum | n = 114 | | n = 113 | |
| PSA at 1 year | | | | |
| PSA response,^{g} n (%) | 28 | (24.6) | 78 | (69.0) |
| OR^{a} (95% CI)^{b} | 0.1 (0.08, 0.26) | | | |
| p^{c} | <0.001 | | | |
| Incidence of a secondary rise in serum | n = 100 | | n = 83 | |
| PSA at 2 years | | | | |
| PSA response,^{g} n (%) | 92 | (92.0) | 77 | (92.8) |

| | |
|---|---|
| OR^{a} (95% CI)^{b} | 1.1 (0.37, 3.53) |
| p^{c} | 0.81 |

^{a}Calculated using an exact logistic regression model with treatment group, stratification factors, age, race, and time since prostate cancer diagnosis as fixed effects, and study site and patient as random effects;
^{b}Calculated based on exact binomial distribution;
^{c}Calculated based on exact binomial distribution from the logistic regression model;
^{d}Analyzed using a mixed model repeated measures model, with treatment group, stratification factors, visit, visit-by-treatment, and baseline score as fixed effects, and study site and patient as random effects;
^{e}Most recent biopsy taken during the 6 months prior to screening;
^{f}Holm-Bonferroni used to adjust for multiplicity; ^{g}Defined as a secondary rise in serum PSA ≥25% of baseline, or ≥25% above nadir, or an absolute increase of ≥2 ng/mL
AS, active surveillance; CI, confidence interval; LS, least squares; OR, odds ratio; PSA, prostate-specific antigen; SE, standard error.

Enzalutamide significantly increased the odds of a negative biopsy at one year versus AS; the proportion of patients with negative biopsies was greater with enzalutamide than with AS at two years, although the difference between arms was not statistically significant (Table 3). The proportion of enzalutamide patients who had a negative biopsy after the first year of the study was 35.1% (40/114) versus 14.2% (16/113) patients in the active surveillance group. The odds ratio (95% CI) was 3.5 (1.76, 6.92), and the P-value was <0.0001. Of those patients who entered the one-year follow-up, 66 patients in the enzalutamide group and 50 patients in AS group reported biopsy results. The second-year proportions were 19.0% (19/100) and 12.0% (10/83) of patients in enzalutamide and active surveillance groups, respectively. The odds ratio (95% CI) were 1.6 (0.66, 4.00), and the P-value was 0.289 for enzalutamide vs. active surveillance patients.

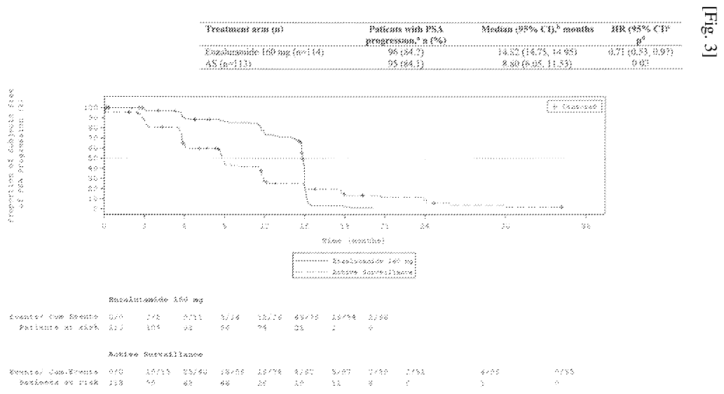

Enzalutamide significantly delayed time to PSA progression by 6 months versus AS (FIG. 3). Results were potentially confounded by the effect of enzalutamide on PSA and the definition of progression being a secondary rise in serum PSA ≥25% above baseline or nadir, resulting in a potential bias towards AS. PSA rebound was demonstrated in the enzalutamide treatment arm following cessation of treatment (observed at month 15 onwards).

Of the 227 patients, 84.2% (96/114) of the enzalutamide patients experienced PSA progression versus 84.1% (95/113) of patients from the active surveillance group. The median time to PSA progression for each of the treatment groups is shown in Table 4. The hazard ratio (95% CI) was 0.714 (0.525, 0.972), favoring enzalutamide vs. active surveillance patients with a statistically significant p-value of 0.032.

TABLE 4
Kaplan-Meier and Cox Regression
for Time to PSA Progression -FAS

| | enzalutamide | Active Surveillance |
|---|---|---|
| Analysis Set-FAS | N = [114] | N = [113] |
| No. of Events -n (%) | 96 (84.2) | 95 (84.1) |
| No. Censored - n (%) [1] | 18 (15.8) | 18 (15.9) |
| Median Time to PSA | 14.82 (14.75, | 8.8 (6.05, |
| Progression (95% CI) | 14.95) | 11.53) |

| | |
|---|---|
| Hazard Ratio (95% CI) [2] | 0.714 (0.525, 0.972) |
| P-Value [3] | 0.032 |

[1] Patients with no PSA progression (secondary rise in serum PSA >25% of baseline or >25% above nadir or absolute increase >2 ng/mL) at the time of trial completion, discontinuation, or death were censored at the last assessment date. Additionally, patients switching therapy during the study were censored at the time of the initial therapy switch, and patients discontinuing therapy were censored at the time of study discontinuation.
[2] Hazard ratio and 95% CI for hazard ratio are based on a Cox regression model assuming proportional hazards with treatment, prostate cancer risk (low vs. intermediate) and type of biopsy (mpMRI targeted vs. non mpMRI targeted), baseline variables such as age, race, and time since prostate cancer diagnosis as fixed effects; and random effect of site. A hazard ratio less than 1 indicates a reduction in hazard rate in favor of enzalutamide 160 mg and vice versa. Patients with no PSA progression (secondary rise in serum PSA >25% of baseline or >25% above nadir or absolute increase >2 ng/mL) at the time of trial completion, discontinuation, or death were censored at the last assessment date. Additionally, patients switching therapy during the study were censored at the time of the initial therapy switch, and patients discontinuing therapy were censored at the time of study discontinuation.
[3] P-value is from a 2-sided, log-rank test.

Enzalutamide significantly reduced the odds of a secondary rise in serum PSA at one year versus AS, but not at two years (Table 3).

There was a statistically significant reduction in mean percentage of cancer-positive cores at one year with enzalutamide versus AS but no statistically significant difference between treatment arms at two years (Table 3). However, within the enzalutamide arm, there was a statistically significant reduction of 6.7% between baseline and year two in the mean percentage of cancer-positive cores.

Safety Variables

Adverse Events

As expected, the incidence of Drug-Related Adverse Events (AEs) were reported in 92.0% (103/112) of the enzalutamide patients during the treatment period plus 30 days. Of the patients on active surveillance, 54.9% (62/113) experienced an adverse event from randomization until date completed 12 months on study plus 30 days. The most common Drug-Related AE seen in the enzalutamide group was fatigue (52.7%). Adverse events which occurred in ≥3.5% of enzalutamide patients were fatigue (55.4%), gynecomastia (36.6%), nipple pain (30.4%), breast tenderness (25.9%), and erectile dysfunction (17.9%). The only AE that occurred in ≥3.5% of the patients on active surveillance was hypertension (7.1%). Table 5A shows the most common AEs. Table 5B shows an overview of the AEs. Observed AE data were consistent with the known safety profile of enzalutamide.

TABLE 5A
The Most Common (>=7.0% of Subjects) Adverse Events-SAF

| | | |
|---|---|---|
| Adverse Event | enzalutamide | Surveillance |
| (MedDRA V5.0) | N = [112] | N = [113] |

| | | | | |
|---|---|---|---|---|
| Fatigue | 62 | (55.4%) | 4 | (3.5%) |
| Gynecomastia | 41 | (36.6%) | 2 | (1.8%) |
| Nipple Pain | 34 | (30.4%) | 0 | (0%) |
| Breast Tenderness | 29 | (25.9%) | 1 | (0.9%) |
| Erectile Dysfunction | 20 | (17.9%) | 2 | (1.8%) |
| Alopecia | 11 | (9.8%) | 0 | (0%) |
| Libido Decreased | 9 | (8.0%) | 1 | (0.9%) |
| Hypertension | 8 | (7.1%) | 8 | (7.1%) |

Descending in incidence of Total Group by System Organ Class, and within that descending order by Preferred Term.

TABLE 5B
Overview of AEs

| | During 1-year treatment | | During 1-year follow-up | | During 1-year continued | |
|---|---|---|---|---|---|---|
| | period^{a} | | period^{b} | | follow-up period^{c} | |
| | Enzalutamide | | Enzalutamide | | Enzalutamide | |
| | 160 mg | AS | 160 mg | AS | 160 mg | AS |
| n (%) | n = 112 | n = 113 | n = 112 | n = 113 | n = 112 | n = 113 |
| AEs | 103 (92.0) | 62 (54.9) | 44 (39.3) | 26 (23.0) | 16 (14.3) | 12 (10.6) |
| SAEs | 9 (8.0) | 5 (4.4) | 8 (7.1) | 3 (2.7) | 4 (3.6) | 2 (1.8) |
| Drug-related AEs | 99 (88.4) | N/A | N/A | N/A | N/A | N/A |
| Drug-related SAEs | 3 (2.7) | N/A | N/A | N/A | N/A | N/A |
| Drug-related AEs leading to | 8 (7.1) | N/A | N/A | N/A | N/A | N/A |
| discontinuation of study | | | | | | |
| drug | | | | | | |
| TEAEs ≥ grade 3^{d} | 11 (9.8) | 10 (8.9) | N/A | N/A | N/A | N/A |
| Drug-related AEs ≥ grade 3^{d} | 6 (5.4) | N/A | N/A | N/A | N/A | N/A |
| AEs leading to death | 1 (0.9) | 0 | 0 | 0 | 2 (1.8) | 0 |

^{a}From date of first dose (enzalutamide) or randomization (AS) until date discontinued or completed 12 months on study + 30 days;
^{b}From date completed 12 months on study + 31 days until date discontinued or completed 24 months on study;
^{c}From date completed 24 months on study + 1 day until date discontinued or completed study;
^{d}AEs graded based on NCI-CTCAE version 4.03
AE, adverse event;
AS, active surveillance;
N/A, not applicable;
NCI-CTCAE, National Cancer Institute Common Terminology Criteria for Adverse Events;
SAE, serious adverse event;
TEAE, treatment-emergent adverse event

after the patient discontinued the treatment period due to non-compliance with the enzalutamide treatment; that patient died by homicide within the 1-year treatment period. The other two deaths (by intracranial hemorrhage and by metastatic cholangiocarcinoma) occurred while patients were in their continued follow-up treatment period. None of the deaths occurred while patients were treated with enzalutamide.

### Health-Related Quality of Life

The effects of enzalutamide treatment on health-related quality of life (HRQoL) were assessed using patient-reported outcomes (PROs). PROs were assessed at baseline and during follow-up using the Brief Fatigue Inventory, Expanded PC (Prostate Cancer) Index Composite (EPIC), 12-Item Short-Form Survey (SF-12), and Memorial Anxiety Scale for PC (MAX-PC).

Fatigue at baseline was mild (≤3) and remained so in the enzalutamide and AS groups through end of treatment (month 12) and end of follow-up (month 24) (Table 6). There was no dysfunction or bother in the hormonal and urinary components of the EPIC score in either group, although a clinically meaningful effect was observed on sexual function in the enzalutamide-treated group, apparent from 6 months (33.4±22.2 vs. 56.7±23.4). The SF-12 mental component showed no impact in either group, although an increased physical impairment was evident in patients receiving enzalutamide at month 12, resolving by month 24 (Table 5). The MAX-PC showed no PC-related anxiety in either group.

TABLE 6
Results of PROs During Follow-Up
Mean (SD) PROs during follow-up

| | Baseline | | Month 12 | | Month 24 | |
|---|---|---|---|---|---|---|
| | enzalutamide | AS | enzalutamide | AS | enzalutamide | AS |
| BFI | 1.5 | 1.5 | 2.5 | 1.6 | 1.6 | 1.6 |
| | (1.6) | (1.5) | (2.0) | (1.6) | (1.5) | (1.6) |
| EPIC | 61.0 | 58.6 | 30.2 | 54.7 | 47.7 | 59.7 |
| sexual | (22.5) | (23.0) | (23.7) | (22.3) | (25.2) | (18.9) |
| EPIC | 89.4 | 87.2 | 85.2 | 86.8 | 88.3 | 86.5 |
| urinary | (9.4) | (13.7) | (11.1) | (9.9) | (10.4) | (11.6) |
| EPIC | 58.6 | 58.5 | 57.7 | 58.7 | 57.9 | 58.7 |
| hormonal | (2.9) | (2.9) | (6.6) | (3.2) | (4.0) | (3.1) |
| SF-12 | 53.2 | 52.6 | 49.4 | 51.8 | 51.0 | 52.1 |
| physical | (6.7) | (6.6) | (8.2) | (7.0) | (7.4) | (6.6) |
| SF-12 | 54.1 | 54.2 | 52.0 | 52.8 | 54.4 | 52.6 |
| mental | (7.2) | (7.1) | (7.4) | (8.0) | (5.9) | (8.5) |
| MAX-PC | 17.1 | 14.9 | 16.5 | 14.8 | 16.3 | 14.1 |
| | (7.4) | (5.3) | (6.2) | (5.1) | (6.4) | (5.2) |

The results show that treatment with enzalutamide is not associated with clinically significant worsening of HRQoL compared with patients receiving AS; however, a clinically relevant effect on sexual function that resolved following treatment cessation was observed.

### CONCLUSIONS

Patients treated with enzalutamide had a 46% reduction in risk of disease progression compared to patients on active surveillance. Median time to disease progression was not reached in either group.

Secondary endpoints included incidence of negative biopsy and time to PSA progression, and the results were in favor of the enzalutamide group. Patients in the enzalutamide group were significantly more likely to have a negative biopsy at one year compared to active surveillance, though this was not seen at two years. The odds of a negative biopsy were 3.5 times higher at one year versus active surveillance. A statistically significant reduction in mean percentage of cancer-positive cores was observed at one year versus active surveillance. Treatment with enzalutamide delayed PSA progression by approximately 6 months, and this delay was statistically significant.

Safety was assessed among the secondary endpoints. Not surprisingly, treated patients exhibited more adverse events than patients on active surveillance, in particular gynecomastia, breast tenderness, and nipple pain were noted. No unexpected adverse events were encountered when patients were in the treatment period. Mortality was higher among the enzalutamide group, however deaths did not occur while patients were being treated with enzalutamide.

Results are summarized in Table 7.

TABLE 7
Summary of Results

| | Enzalutamide^{a} | Active surveillance^{a} |
|---|---|---|
| Time to prostate cancer | n = 114 | n = 113 |
| progression^{b} | | |
| Events, n (%) | 32 (28.1) | 42 (37.2) |
| Median (95% CI), months | NR (35.5, | NR (24.2, |
| | NR) | NR) |
| HR^{d} (95% CI) | 0.54 (0.33, 0.89) | |
| p | 0.02* | |
| Negative biopsy at 1 year | n = 114 | n = 113 |
| Events, n (%) | 40 (35.1) | 16 (14.2) |
| OR^{e} (95% CI) | 3.50 (1.76, 6.92) | |
| p | <0.0001*** | |
| Negative biopsy at 2 years | n = 100 | n = 83 |
| Events, n (%) | 19 (19.0) | 10 (12.0) |
| OR^{e} (95% CI) | 1.60 (0.66, 4.00) | |
| p | 0.29 | |
| Time to PSA progression^{c} | n = 114 | n = 113 |
| Events, n (%) | 96 (84.2) | 95 (84.1) |
| Median (95% CI), months | 14.8 (14.8, | 8.8 (6.1, |
| | 15.0) | 11.5) |

| | |
|---|---|
| HR^{d} (95% CI) | 0.71 (0.53, 0.97) |
| p | 0.03* |

CI, confidence interval; ECOG, Eastern Cooperative Oncology Group; GS, Gleason Score; HR, hazard ratio; NR, not reached; OR, odds ratio; PSA, prostate-specific antigen.
^{a}Randomization stratified by risk (low vs intermediate) and type of biopsy performed (transrectal ultrasound-guided prostate biopsy [mpMRI targeted vs non-mpMRI targeted]). Low risk defined as T1c-T2a, PSA <10, N0, M0, GS ≤6, ECOG status ≤2, and estimated life expectancy >5 years. Intermediate risk defined as T2b-T2c, PSA <20, N0, M0, GS ≤7 (3 + 4 pattern only), ECOG status ≤2, and estimated life expectancy >5 years; ^{b}Pathological or therapeutic prostate cancer progression. Pathological progression defined as an increase in primary or secondary Gleason pattern by >1 or >15% increase in cancer positive cores. Therapeutic progression defined as the earliest occurrence of primary therapy for prostate cancer (prostatectomy, radiation, focal therapy, or systemic therapy);
^{c}Defined as a ≥25% increase in serum PSA from baseline, a ≥25% increase above nadir, or an absolute increase of >2 ng/mL;
^{d}Cox regression model;
^{e}Exact logistic regression model.
*p < 0.05;
***p < 0.001

1. A method of reducing the risk of prostate cancer progression in a patient with a histologically proven prostate cancer characterized as low or intermediate risk for which no other treatment for prostate cancer is indicated, comprising administering enzalutamide monotherapy to the patient in an amount and for a period of time clinically proven effective in reducing the risk of prostate cancer progression.

2. The method of claim 1, wherein the risk of prostate cancer progression in the patient is reduced relative to a control population of patients under surveillance, wherein patients in the control population have histologically proven prostate cancers characterized as low or intermediate risk for which no other treatment for prostate cancer is indicated, and wherein patients in the control population do not receive a therapy for prostate cancer.

3. The method of claim 1, wherein the life expectancy of the patient and the life expectancies of patients in the control population is >5 years.

4. The method of claim 1, wherein the life expectancy of the patient and the life expectancies of patients in the control population is ≤5 years.

5. The method of claim 1, wherein the prostate cancer of the patient and the prostate cancers of the control population are characterized as low risk.

6. The method of claim 5, wherein low risk is defined by one or more of a stage identification of T1c-T2a, prostate-specific antigen (PSA) level <10 ng/mL, a Gleason score of ≤6, and an Eastern Cooperative Oncology Group (ECOG) status of ≤2.

7. The method of claim 1, wherein the prostate cancer of the patient and the prostate cancers of the control population are characterized as intermediate risk.

8. The method of claim 7, wherein intermediate risk is defined by one or more of a stage identification of T2b-T2c, a PSA level <20 ng/mL, a Gleason score of ≤7 (3+4 pattern), and an ECOG status ≤2.

9. The method of claim 1, wherein the prostate cancer progression comprises pathological prostate cancer progression.

10. The method of claim 9 wherein the pathological prostate cancer progression comprises an increase in primary or secondary Gleason pattern by >1 or a >15% increase in cancer positive cores.

11. The method of claim 1, wherein the prostate cancer progression comprises therapeutic prostate cancer progression.

12. The method of claim 11, wherein the therapeutic prostate cancer progression comprises the earliest occurrence of a further therapy for prostate cancer.

13. The method of claim 12, wherein the further therapy is selected from the group consisting of androgen deprivation therapy, prostatectomy, radiation, focal therapy, and systemic therapy.

14. The method of claim 1, wherein the period of time is one year.

15. The method of claim 1, wherein the likelihood of the patient having a negative biopsy after one year of enzalutamide monotherapy is increased at least three-fold relative to the likelihood of patients in the control population having negative biopsies after one year of active surveillance.

16. The method of claim 1, wherein PSA progression in the patient is delayed by six months relative to PSA progression of patients in the control population.

17. The method of claim 1, wherein the enzalutamide monotherapy is administered at a daily dose of 160 mg.

18. The method of claim 1, wherein a strong CYP3A4 inducer is co-administered to the patient and wherein the enzalutamide monotherapy is administered at a daily dose of 240 mg.
